(19) United States
(12) Patent Application Publication
(10) Pub. No.: US 2026/0257032 A1
(43) Pub. Date: Sep. 3, 2026

(54) MEDICAL TUBE AND/OR CORD SUPPORT METHODS AND APPARATUS

(71) Applicant: TalexMedical, Malvern, PA (US)

(72) Inventors: Bryan Griffiths, Atglen, PA (US); Michael Mazzio, Schwenksville, PA (US)

(21) Appl. No.: 19/553,311

(22) Filed: Feb. 28, 2026

Related U.S. Application Data
(60) Provisional application No. 63/765,519, filed on Feb. 28, 2025.

(51) Int. Cl. A61M 16/04; A61M 16/08

(52) U.S. Cl. CPC A61M 16/0461; A61M 16/0816; A61M 2209/088; A61M 2210/06; A61M 2210/1032

(57) ABSTRACT

Herein are described methods and apparatus to affix conduits to a patient's skin and hold the conduits in place relative to the patient. These methods and apparatus being useful in surgical and medical care settings.

### CROSS REFERENCE TO RELATED APPLICATIONS

This application claims priority to U.S. Provisional Application No. 63/765,519 , filed 28 Feb. 2025, the disclosure of which is hereby incorporated by reference in its entirety.

### FIELD OF THE INVENTION

This invention relates to methods and apparatus that are affixed to the patient's skin and hold conduits in place relative to the patient.

### BACKGROUND OF THE INVENTION

Current methods of securing a nasotracheal tube used for mechanical ventilation of patients during surgery and recovery do not reliably keep the nasotracheal tube and the ventilator connections in place. This leads to the nasotracheal tube inadvertently being pulled out, or the tubing moving out of place and interfering with the procedure.

In view of the above, it would be beneficial to develop an apparatus to securely hold a ventilator tube connector (and/or other medical conduits) securely to a patient and methods of using the same.

### BRIEF SUMMARY OF THE INVENTION

In an aspect, there are described a novel conduit-securing apparatuses designed to securely hold at least one conduit to a patient.

In another aspect, the novel conduit-securing apparatuses are designed to adhere to a patient's forehead.

In another aspect, there are described novel conduit-securing apparatuses, comprising:
(a) optionally, an apparatus body, comprising:
   i. first conduit holder;
   ii. a first apparatus surface;
   iii. a second apparatus surface; and,
(b) optionally, a substrate, which is designed to adhere to a patient (e.g., a patient's forehead);
   wherein:
      at least one of the apparatus body and attachment substrate is present;
      when the substrate is absent, the second apparatus surface is designed to adhere to a dermal surface of a patient (e.g., a patient's forehead); and,
      when the substrate is present, the second apparatus surface is designed to interface with the substrate.

In another aspect, there are described novel methods of adhering the conduit-securing apparatus to a patient.

In another aspect, there are described novel methods of adhering conduits to the novel conduit-securing apparatuses described herein.

The specific aspects described below relate to method and apparatus to securely hold the ventilator tube connector securely to the patient, however the general principals described can be applied broadly to securely holding a variety of conduits to the patient.

### BRIEF DESCRIPTION OF THE DRAWINGS

FIG. 1A shows a top, side-angled perspective view of apparatus body 1.

FIG. 1B shows a bottom, side-angled view of apparatus body 1.

FIG. 2A shows a top, side-angled perspective view of attachment substrate 20.

FIG. 2B shows a top, side-angled perspective view of attachment substrate 20 with part of attachment substrate 20 folded back to see a first substrate surface 21.

FIG. 3A illustrates a front view showing the placement of attachment substrate 20 with the first substrate surface 21 conformed to, and in contact with a patient's forehead 30.

FIG. 3B illustrates a front view showing the combination of attachment substrate 20 and apparatus body 1 in contact with a patient's forehead 30.

FIG. 3C illustrates a front view showing the apparatus described in FIG. 3B with a ventilation tube connector 32 and a nasotracheal tube 31 (a conduit)

FIG. 4 illustrates a front view showing apparatus body 1 secured directly to a patient's forehead 30 as well as a nasotracheal tube 31 (a conduit), ventilation tube 33 (a conduit), and ventilation tube connector 32.

FIG. 5 illustrates a front view showing a ventilation tube connector 32 secured to attachment substrate 20 as well as a nasotracheal tube 31, ventilation tube 33, and ventilation tube connector 32.

FIGS. 6A-6B illustrate a side view and top view, respectively, of a universal corrugated connector 32 secured to first conduit holder 2 via mating feature 7 and recess 8.

FIG. 6C illustrates a top view of a nasotracheal tube 31 secured to first conduit holder 2 via mating feature 7 and recess 8.

### DETAILED DESCRIPTION OF ASPECTS

While the disclosed technology may have aspects in many different forms, there is shown in the drawings and will herein be described several specific aspects with the understanding that the present disclosure is to be considered as an exemplification of the principles of the technology and is not intended to limit the technology to the aspects illustrated. The terminology used herein is for the purpose of describing particular aspects of the present invention and is not intended to limit the technology.

Conduit(s) as used herein refer to tubes, cords, cables, wires, or other similar devices used for medical treatment and/or care of a patient. During some treatments and/or procedures it is necessary to have at least one conduit (one or more conduits) connected to the patient. It is often necessary to secure these conduits to ensure that they stay in place and out of the way to allow access to the patient during procedures and or treatments. Management of these conduits to ensure that they stay in place and do not interfere with other devices or with access to the patient can be a challenge.

Therefore, in an aspect, there are described novel methods and apparatus(es) to secure at least one conduit (or a plurality of conduits) to a patient.

In some aspects, the methods and apparatus are configured to allow for the effective management of these conduits to ensure that they stay in place relative to the patient.

In some aspects, when the conduit(s) is a tube(s), it(they) can be used to transport gases, liquids, or other materials to and from the patient.

In some aspects, the conduit(s) is (or includes) a cord(s), cable(s), and/or wire(s), which can be used to support a number of other medical technologies. Securing these various conduits to a patient would be helpful in a surgical, post-op, and/or hospital setting.

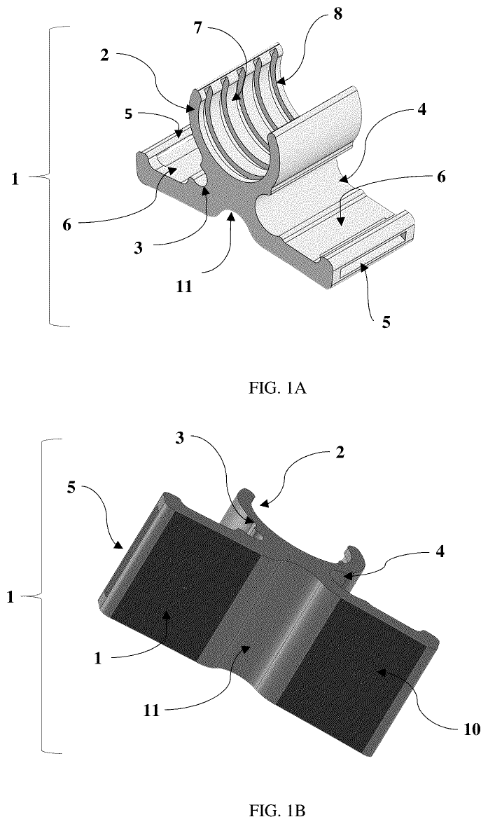

Some aspects of the present invention are illustrated in FIGS. 1-6C. These figures are intended to further clarify the present invention and are only exemplary in nature.

Numbers in FIGS. 1-6C correspond to the following structures/features:
1) Apparatus body.
2) First conduit holder.
3) Second conduit holder.
4) Third conduit holder.
5) Attachment point.
6) First apparatus surface.
7) Mating feature.
8) Recess.
10) Second apparatus surface.
11) By-pass feature.
20) Attachment substrate
21) First substrate surface.
22) Second substrate surface.
30) Patient's forehead.
31) Nasotracheal tube.
32) Tube connector.
33) Tube.
34) Interlocking surface.

### APPARATUS

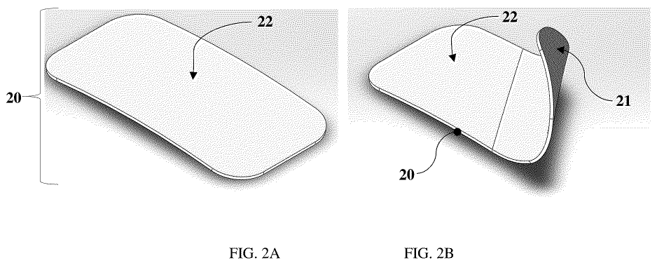

In another aspect, there is described a novel conduit-securing apparatus (see FIGS. 1A-2B), comprising:
(a) optionally, apparatus body 1, comprising:
   i) first conduit holder 2;
   ii) a first apparatus surface 6;
   iii) a second apparatus surface 10; and,
(b) optionally, substrate 20, which is designed to adhere to a patient (e.g., a patient's forehead);
   wherein:
      at least one of apparatus body 1 and substrate 20 is present;
      when substrate 20 is absent, second apparatus surface 10 is designed to adhere to a dermal surface of a patient (e.g., a patient's forehead); and,
      when substrate 20 is present, second apparatus surface 10 is designed to interface with substrate 20.

In another aspect, apparatus body 1 is present and substrate 20 is absent

In another aspect, apparatus body 1 is absent and substrate 20 is present

In another aspect, there the first conduit holder 2, comprises: a mating feature 7.

In some aspects, the first conduit holder 2 is configured to securely hold at least one conduit. In some aspects, 2 is configured to securely hold a plurality of conduits. In some aspects, 2 is in the shape of a half-cylinder (see FIG. 1A).

In some aspects, apparatus body 1, comprises: a plurality of conduit holders (e.g., 2, 3, and 4) to hold multiple conduits. In some aspects, each of the plurality of conduit holders is in the shape of a half-cylinder.

In some aspects, the plurality of conduit holders (e.g., 2, 3, and 4) can hold different sizes and/or diameters of conduits.

Examples of mechanism to secure and release conduits from at least one of the conduit holders (e.g., 2, 3, and 4) include articulating clamps, zip ties, straps, etc. Further examples include the use of adhesives and/or a matrix material that would conform to the conduit secure it to the conduit holder(s). The adhesive and/or matrix material would solidify by cooling or otherwise setting up. Other examples of ways of securing a conduit(s) to the apparatus include: the use of magnetic attachments, straps, ratcheting features, clamps, adhesives, or interlocking features such as snaps, or hook and loop, or other such mechanisms.

In some aspects, the first conduit holder 2 is configured to securely hold at least one conduit connector (e.g., tube connector 32). In some aspects, the conduit connector is designed to secure a conduit.

In some aspects, first apparatus surface 6 can be used to secure or place other devices on apparatus body 1.

In another aspect, mating feature 7, comprises: a surface shaped and sized to mate with at least one conduit (or a conduit connector). In some aspects, 7 is configured to mate with a plurality of conduits.

In another aspect, apparatus body 1, further comprises: at least one recess 8, which is configured to accommodate at least one protrusion in the conduit to be held.

In another aspect, apparatus body 1, further comprises: a plurality of recesses 8, which are configured to accommodate at least one protrusion in the conduit to be held.

Nasotracheal tubes, regardless of the size, have the same connector size at one end. This connector fits a universal corrugated connector, which comprises a flange on one end.

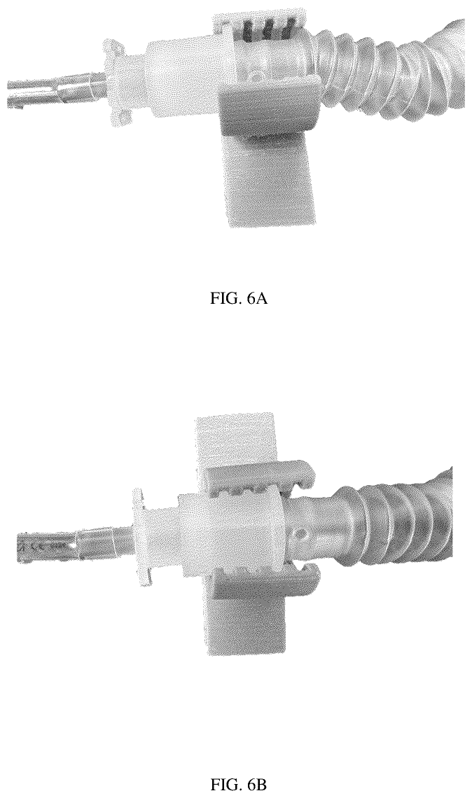

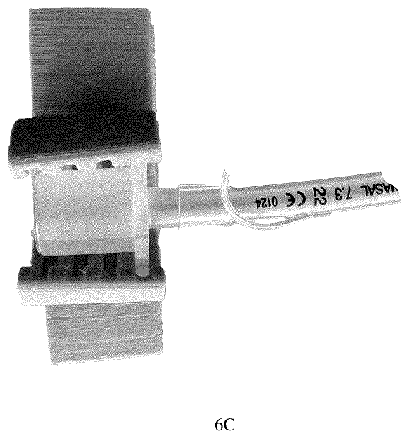

In some aspects, first conduit holder 2 is configured to receive a universal corrugated connector flange (FIGS. 6A-6B).

In some aspects, first conduit holder 2 is configured to receive a nasotracheal tube (without the use of a universal corrugate connector)(FIG. 6C).

In some aspects, mating feature 7 is configured to receive a universal corrugated connector flange (FIGS. 6A-6B).

In some aspects, mating feature 7 is configured to receive a nasotracheal tube (without the use of a universal corrugate connector) (FIG. 6C).

In some aspects, recess 8 is configured to receive a universal corrugated connector flange (FIGS. 6A-6B).

In some aspects, recess 8 is configured to receive a nasotracheal tube (without the use of a universal corrugate connector) (FIG. 6C).

In some aspects, apparatus body 1, further comprises: at least one attachment point 5, which is configured to allow apparatus body 1 to be mechanically secured to the patient (e.g., using straps or bands). As can been seen in FIG. 1A, attachment point 5 comprises an opening which can allow a strap, band, or similar material to pass through it (e.g., to be wrapped around or affixed apparatus body 1).

In some aspects, attachment point 5 is configured to allow for other devices to be attached to it.

In some aspects, attachment point 5 is configured to both allow for other devices to be attached to it and to allow for apparatus body 1 to be attached to a patient.

In some aspects, the second apparatus surface 10 (see FIG. 1B) is shaped to conform to a patient's anatomy (e.g., curved to mirror a patient's forehead).

In some aspects, 10 is further configured to interface with attachment substrate 20 (see FIGS. 2A-2B) or directly with the patient to secure apparatus body 1 in place.

In some aspects, second apparatus surface 10, comprises: a conforming material (e.g., foam or other pliable material) to allow it to form to the patient's anatomy.

In some aspects, surfaces 10 and 22, comprise: interlocking materials (e.g., hook and loop or other mechanisms described herein). This aspect allows apparatus body 1 to be repositioned as needed on attachment substrate 20.

In some aspects, attachment substrate 20 (see FIGS. 2A-FIG. 2B) comprises a material that be trimmed or otherwise adapted to size, to best fit the patient's anatomy.

In some aspects, apparatus body 1, further comprises: by-pass feature 11, which is designed to allow apparatus body 1 to be placed over the anatomy of a patient and/or another device (e.g., another conduit). Examples of other devices include a pulse oximeter, which is often attached to the patient in the same area as would be desirable to attach apparatus body 1. Feature 11 would allow the apparatus body 1 to be co-located without disrupting the function of the other device.

In some aspects, second apparatus surface 10, comprises: an adhesive (e.g., a biocompatible adhesive, which are publicly available from companies including 3M and Adhesives Research).

In some aspects, second apparatus surface 10, comprises:
(a) an adhesive; and,
(b) a release liner;
   wherein the release liner protects the adhesive from its environment and can be removed when a practitioner wants to expose the adhesive to the atmosphere (e.g., to adhere attachment substrate 20 to a patient).

In some aspects, attachment substrate 20 is present (see FIGS. 2A and 2B) and, comprises:
(a) a First Substrate Surface 21; and,
(b) a second substrate surface 22.

In some aspects, first substrate surface 21, comprises: an adhesive (e.g., a biocompatible adhesive).

In some aspects, first substrate surface 21, comprises:
(a) an adhesive; and,
(b) a release liner;
   wherein the release liner protects the adhesive from its environment and can be removed when a practitioner wants to expose the adhesive to the atmosphere (e.g., to adhere attachment substrate 20 to a patient).

In some aspects, the adhesive is chosen such that it is capable of securing both attachment substrate 20 and apparatus body 1 (or just the Body 1 if 20 is absent)

In some aspects, the adhesive causes minimal irritation to the patient's skin and can be removed without substantial damage to the patient's skin.

In some aspects, second apparatus surface 10, comprises an even layer of adhesive across the substantially (e.g., 80, 85, 90, 95, 99, to 100% coverage) the entire surface of 10.

In some aspects, first substrate surface 21, comprises an even layer of adhesive across the substantially (e.g., 80, 85, 90, 95, 99, to 100% coverage) the entire surface of 21.

In some aspects, second substrate surface 22 is designed to interface with second apparatus surface 10 and/or allow other conduits to be secured to it.

In some aspects, attachment substrate 20 is present (see FIGS. 2A and 2B) and, comprises:
(a) a first substrate surface 21, which comprises: an adhesive (described herein); and,
(b) a second substrate surface 22, which comprises: an interlocking material (e.g. a hook or loop or other mechanisms described herein).

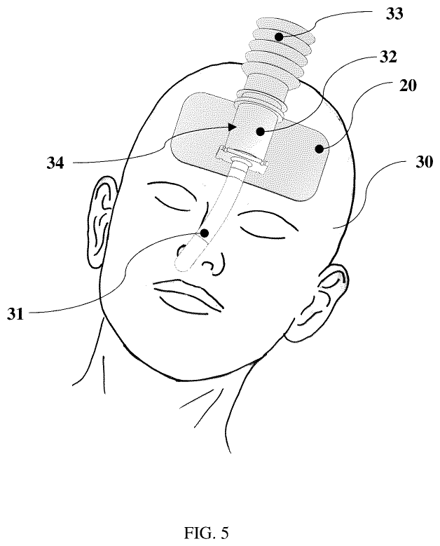

In some aspects, the conduit is at least partially wrapped with an interlocking material that locks with the interlocking material of surface 22 (e.g., a hook or loop or other mechanisms described herein)(see FIG. 5). This allows for the conduit to be secured directly to surface 22. This also allows the conduit to be easily repositioned as needed.

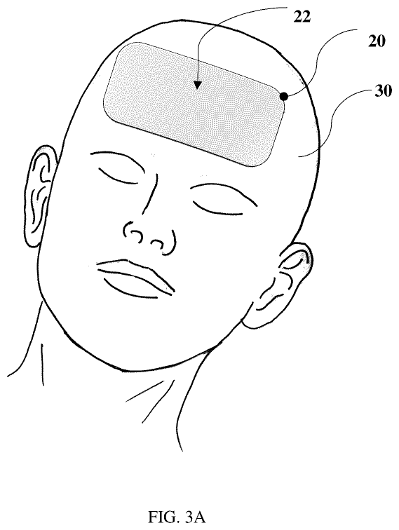

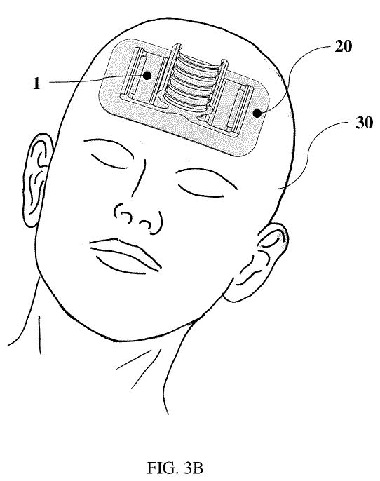

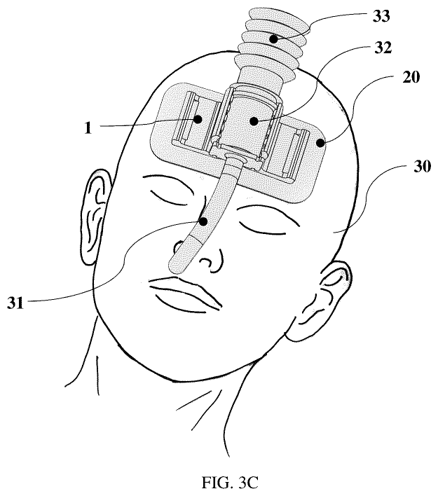

FIG. 3A shows the placement of attachment substrate 20 with the first substrate surface 21 conformed to and in contact with a patient's forehead 30.

FIG. 3B shows apparatus body 1 when the attachment substrate 20 is present and where first substrate surface 21 is conformed to and in contact with a patient's forehead 30 and second apparatus surface 10 is secured to second substrate surface 22.

In some aspects, second substrate surface 22, comprises: a conforming material (e.g., foam or other pliable material) to allow it to form to the patient's anatomy.

In some aspects, second substrate surface 22 (similar to first apparatus surface 6), allows for the attachment of other devices to attachment substrate 20.

In some aspects, apparatus body 1, further comprises: a tube connector 32.

In some aspects, tube connector 32 is secured to first conduit holder 2.

In some aspects, the tube connector 32 is a ventilation tube connector.

In some aspects, apparatus body 1, further comprises: a tube connector 32 (e.g., a ventilation tube), a nasotracheal tube 31 (which is connected to the ventilation tube connector 32), and a ventilation tube 33 (which is connected to the ventilation tube connector 32).

FIG. 3C shows an aspect where the apparatus described in FIG. 3B with a tube connector 32 (e.g. ventilation tube) secured to the apparatus body 1 via first conduit holder 2. FIG. 3C also shows an aspect where nasotracheal tube 31, which can be placed in a patient, connected to the tube connector 32, and a ventilation tube 33 is connected to the tube connector 32.

In some aspects, attachment substrate 20 is absent. In these aspects, apparatus body 1 can be directly adhered to a patient via second apparatus surface 10.

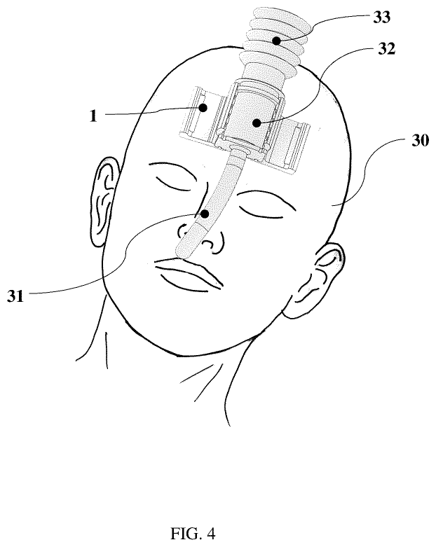

FIG. 4 shows an aspect where surface 10 of apparatus body 1 is secured directly to the patient's forehead 30. FIG. 4 also shows an aspect where ventilation tube connector 32 is secured to the apparatus body 1 via first conduit holder 2. FIG. 4 also shows an aspect where nasotracheal tube 31, which can be placed in a patient, connected to the tube connector 32, and a ventilation tube 33 is connected to the tube connector 32.

In some aspects, tube connector 32, comprises: an interlocking surface 34. Examples of such surfaces include a hook and loop (or other mechanisms described herein).

In another aspect, apparatus body 1 is absent, attachment substrate 20 is present (see FIG. 5), and attachment substrate 20, comprises:
(a) a first substrate surface 21, which comprises: an adhesive designed to adhere to a dermal surface of a patient (e.g., a patient's forehead); and,
(b) a second substrate surface 22, which comprises: an interlocking material.

In some aspects, the second substrate surface 22 allows for the attachment of devices to attachment substrate 20 (e.g., conduit(s), conduit holder(s), and other medical apparatus).

In some aspects, the first substrate surface 21, further comprises: a tube connector 32.

FIG. 5 shows an aspect where tube connector 32 (e.g., ventilation tube) has an interlocking surface 34 such as a hook and loop (or other mechanisms described herein), which is secured to the second substrate surface 22 (not identified) of attachment substrate 20. FIG. 5 also shows an aspect where nasotracheal tube 31, which can be placed in a patient, connected to the tube connector 32, and a ventilation tube 33 is connected to the tube connector 32.

In some aspects, the sizes of apparatus body 1 and attachment substrate 20 can be adjusted to fit patient anatomy and conduit sizes. In some aspects, the apparatus is sized to receive conduits from <1 mm to >20 mm in diameter. In some aspects, the apparatus is sized to receive a nasotracheal tube (or nasotracheal tube connector, in particular the flange present thereon), an IV line (e.g., on a patient's arm), an electrical wire or plurality of wires, or a combination thereof.

In some aspects, the apparatus is configured to attach to a patient's forehead, a patient's arm (e.g., bicep area), shoulder, chest (e.g., sternum), thigh, or other body part/area that would benefit from securing a conduit or conduits.

In some aspects, patient is a human.

### METHODS

In some aspects, there are described novel methods of using a conduit-securing apparatus described herein.

In some aspects, there is described a method of securing a conduit-securing apparatus described herein to a patient's skin.

In some aspects, the method of securing a conduit-securing apparatus described herein to a patient's skin, comprises:
contacting the adhesive present on second apparatus surface 10 to a patient's skin (see FIG. 4).

In some aspects, the method of securing a conduit-securing apparatus described herein to a patient's skin, comprises:
(a) exposing the adhesive present on second apparatus surface 10; and,
(b) contacting the exposed adhesive to a patient's skin (see FIG. 4).

In some aspects, the method of securing a conduit-securing apparatus described herein to a patient's skin, comprises:
contacting the adhesive present on first substrate 21 to a patient's skin (see FIG. 3A).

In some aspects, the method of securing a conduit-securing apparatus described herein to a patient's skin, comprises:
(a) exposing the adhesive present on first Subsurface 21; and,
(b) contacting the exposed adhesive to a patient's skin (see FIG. 3A).

In some aspects, the adhesive is a biocompatible adhesive.

In some aspects, after the first substrate 21 has been adhered to the patient's skin, the method, further comprises:
(c) securing second apparatus surface 10 (see FIGS. 1A-FIG. 1B) of apparatus body 1 to second substrate surface 22 of attachment substrate 20 (see FIG. 3B).

In some aspects, the method, after adhering the conduit-securing apparatus to a patient, further comprises: securing at least one conduit (or a plurality of conduits) to apparatus body 1. The conduit(s) will be held securely in place as shown in FIG. 3C.

In some aspects, after the first substrate 21 has been adhered to the patient's skin, the method, further comprises:
(d) securing a conduit (or a plurality of conduits) to second substrate surface 22 of attachment substrate 20 (see FIG. 5) via an interlocking material (e.g., hook in loop or other mechanisms described herein).

In some aspects, after the first substrate 21 has been adhered to the patient's skin, the method, further comprises:
(e) securing tube connector 32, which comprises: an interlocking surface 34 to second substrate surface 22 of attachment substrate 20 (see FIG. 5) via an interlocking material (e.g., hook in loop or other mechanisms described herein); and,

In these aspects, each of the second substrate surface 22 and the conduit(s), comprises a compatible interlocking material (e.g., hook and loop or other mechanisms described herein). This allows for the conduit to be secured directly to surface 22. This also allows the conduit to be easily repositioned as needed.

Numerous modifications and variations of the present invention are possible considering the above teachings. It is therefore to be understood that within the scope of the appended claims, the invention may be practiced otherwise than as specifically described herein.

What is claimed is:

1. A conduit-securing apparatus, comprising:
(a) optionally, an apparatus body, comprising:
i) first conduit holder;
ii) a first apparatus surface;
iii) a second apparatus surface; and,
(b) optionally, a substrate, which is designed to adhere to a patient (e.g., a patient's forehead);
wherein:
at least one of apparatus body and substrate is present;
when substrate is absent, second apparatus surface is designed to adhere to a dermal surface of a patient (e.g., a patient's forehead); and,
when substrate is present, second apparatus surface is designed to interface with substrate.

2. The conduit-securing apparatus of claim 1, wherein apparatus body is present.

3. The conduit-securing apparatus of claim 1, wherein first conduit holder, comprises: a mating feature.

4. The conduit-securing apparatus of claim 2, wherein the first conduit holder is configured to securely hold at least one conduit or tube connector.

5. The conduit-securing apparatus of claim 2, wherein first apparatus surface is configured to secure other devices on apparatus body.

6. The conduit-securing apparatus of claim 3, wherein mating feature, comprises: a surface shaped and sized to mate with at least one conduit or a conduit connector.

7. The conduit-securing apparatus of claim 3, wherein apparatus body, further comprises:
at least one recess, which is configured to accommodate at least one protrusion in the conduit to be held.

8. The conduit-securing apparatus of claim 7, wherein recess, is configured to receive a universal corrugated connector flange.

9. The conduit-securing apparatus of claim 2, wherein apparatus body, further comprises:
at least one attachment point, which is configured to allow apparatus body to be mechanically secured to the patient.

10. The conduit-securing apparatus of claim 2, wherein the second apparatus surface is configured to interface with attachment substrate or directly with the patient to secure apparatus body in place.

11. The conduit-securing apparatus of claim 2, wherein apparatus body, further comprises:
by-pass feature, which is designed to allow apparatus body to be placed over the anatomy of a patient and/or another device.

12. The conduit-securing apparatus of claim 2, wherein second apparatus surface, comprises: an adhesive.

13. The conduit-securing apparatus of claim 2, wherein attachment substrate is present and, comprises:
(a) a first substrate surface; and,
(b) a second substrate surface.

14. The conduit-securing apparatus of claim 2, wherein first substrate surface, comprises: an adhesive.

15. The conduit-securing apparatus of claim 2, wherein second substrate surface is designed to interface with second apparatus surface and/or allow other conduits to be secured to it.

16. The conduit-securing apparatus of claim 12, wherein the second apparatus surface and, second substrate surfaces comprise: interlocking materials.

17. The conduit-securing apparatus of claim 1, wherein attachment substrate is present and, comprises:
(a) a first substrate surface, which comprises: an adhesive; and,
(b) a second substrate surface, which comprises: an interlocking material.

18. The conduit-securing apparatus of claim 17, wherein apparatus body, further comprises: a tube connector.

19. The conduit-securing apparatus of claim 17, wherein apparatus body, further comprises:
a tube connector;
a nasotracheal tube; and,
and a ventilation tube.

20. The conduit-securing apparatus of claim 1, wherein apparatus body is absent and attachment substrate is present and, attachment substrate comprises:
(a) a first substrate surface, which comprises: an adhesive designed to adhere to a dermal surface of a patient; and,
(b) a second substrate surface, which comprises: an interlocking material.

21. The conduit-securing apparatus of claim 20, wherein the conduit to be secured is at least partially wrapped with an interlocking material that locks with the interlocking material of surface.

22. The conduit-securing apparatus of claim 20, wherein first substrate surface, further comprises: a tube connector, comprising: an interlocking surface.
